(19) United States
(12) Patent Application Publication
(10) Pub. No.: US 2009/0156693 A1
(43) Pub. Date: Jun. 18, 2009

(54) OPHTHALMIC COMPOSITIONS CONTAINING POLYSACCHARIDE/BORATE GELLING SYSTEM

(75) Inventor: Bahram Asgharian, Arlington, TX (US)

Correspondence Address: ALCON, IP LEGAL, TB4-8, 6201 SOUTH FREEWAY, FORT WORTH, TX, 76134, US

(73) Assignee: ALCON, INC., Fort Worth, TX (US)

(21) Appl. No.: 12/390,996

(22) Filed: Feb. 23, 2009

Related U.S. Application Data
(63) Continuation of application No. 11/001,204, filed on Dec. 1, 2004.
(60) Provisional application No. 60/528,646, filed on Dec. 11, 2003.

(51) Int. Cl. A61K 47/36; A61P 27/02

(52) U.S. Cl. 514/777

(57) ABSTRACT

Topical ophthalmic compositions that form a gel or partial gel upon application to the eye are described. The compositions are particularly useful as artificial tears and ocular lubricants, but may also be utilized for the topical delivery of pharmaceutically active compounds to the eye. The compositions contain a polysaccharide/borate gelling system. The polysaccharides that may be utilized contain cis-diol groups and have a structure that is predominately linear, with a slight degree of branching.

### CROSS-REFERENCE TO RELATED APPLICATION

This application is a Continuation (CON) of co-pending U.S. application Ser. No. 11/001,204, filed Dec. 1, 2004, priority of which is claimed under 35 U.S.C. §120, the contents of which are incorporated herein by reference. This application also claims priority under 35 U.S.C. §119 to U.S. Provisional Patent Application No. 60/528,646, filed Dec. 11, 2003, the contents of which are incorporated herein by reference.

### BACKGROUND OF THE INVENTION

The present invention is directed to ophthalmic compositions that form a gel when applied to the eye. The transformation of the compositions from a solution to a gel is based on the presence of a polysaccharide/borate gelling system in the compositions. The compositions are particularly adapted for use as ocular lubricants or artificial tears.

Various types of gelling systems for ophthalmic compositions have been described in the prior art:

The use of a PVA/borate gelling system to form topical ophthalmic gels for delivery of various drugs to the eye is described in U.S. Pat. No. 4,255,415 (Chrai, et al.). However, the '415 patent does not disclose ophthalmic solutions containing PVA and borate that form gels upon application to the eye, nor does it describe the use of such gel-forming solutions as ocular lubricants or artificial tears.

WIPO Publication No. WO 94/10976 (Goldenberg et al.) discloses a low pH PVA-borate delivery system that does go through liquid/gel transition. This system has the disadvantage, however, of limited gelling effects, and only at certain concentrations of PVA depending on the molecular weight of the PVA utilized.

U.S. Pat. No. 4,136,173 (Pramoda, et al.) discloses the use of therapeutic compositions containing xanthan gum and locust bean gum which are administered in liquid form and gel upon instillation. This reference describes a mechanism for transition from liquid to gel involving pH change. pH sensitive gels such as carbomers, xanthan, gellan, and those described above, need to be formulated at or below the pKa of their acidic groups (typically at a pH of about 2 to 5). Compositions formulated at low pH, however, are irritating to the eye.

The use of locust bean gum to form a gel vehicle for ophthalmic drug delivery is described in U.S. Pat. No. 4,136,177 (Lin, et al.). However, the gels described by Lin, et al. are formed at the time of manufacture, rather than upon application to the eye.

U.S. Pat. No. 4,861,760 (Mazuel, et al.) discloses ophthalmic compositions containing gellan gum which are administered to the eye as non-gelled liquids and gel upon instillation due to a change in ionic strength. These systems do not involve the use of small cross-linking molecules, but instead provide gel characteristics due to self cross-linking during ionic condition changes.

Gels involving the cross-linking of polysaccharides with borates are disclosed for use as well fracturing fluids in U.S. Pat. Nos. 5,082,579 (Dawson), 5,145,590 (Dawson), and 5,160,643 (Dawson). These patents describe the use of borates and polysaccharides for industrial oil well excavation.

The use of galactomannans (e.g., guar) in ophthalmic compositions, including ocular lubricant and artificial tear compositions, is described in U.S. Pat. No. 6,583,124 (Asgharian). An ocular lubricant eye drop containing hydroxypropyl guar is sold under the name “SYSTANE™” by Alcon Laboratories, Inc.

The galactomannans described in U.S. Pat. No. 6,583,124 are polysaccharides. Galactomannans have mannan backbones and side chains of galactose. The present invention is directed to the use of other types of polysaccharides to form gels upon application to the eye.

### SUMMARY OF THE INVENTION

The present invention is directed to the ophthalmic compositions that contain a gelling system comprising a polysaccharide and a borate cross linker. The compositions are formulated and manufactured as liquids or partially gelled liquids that thicken to form gels upon application to the eye. The compositions of the present invention are particularly useful as artificial tears or ocular lubricants, but may also be utilized to deliver ophthalmic drugs to the eye.

The polysaccharides utilized in the present invention contain cis-diol groups that are capable of interacting with borates to form gels upon application to the eye and have a structure that is predominately linear with a low degree of branching.

### BRIEF DESCRIPTION OF THE DRAWING(S)

FIG. 1 is a graph showing the viscosity of the composition described in Example 1, as a function of pH.

### DETAILED DESCRIPTION OF THE INVENTION

The compositions of the present invention contain an amount of a polysaccharide/borate gelling system sufficient to form a gel or partial gel upon application of the compositions to the eye.

The polysaccharides utilized in the invention contain cis-diol groups that interact with borates to form gels when subjected to a small shift in pH. The polysaccharides are predominately linear with a low degree of branching, as compared to other polysaccharides that are highly branched polymers (e.g., galactomannans). The preferred polysaccharides contain less than one branched group per five sugar moieties. The cis-diol groups are formed by hydroxyl groups on adjacent carbon atoms that are in a cis configuration (i.e., one carbon in an axial orientation and the other carbon in an equatorial position). The sugar groups have either α or β linkages at the 1,4-position.

The polysaccharides that may be utilized in the present invention include all pharmaceutically acceptable compounds that have the foregoing structural features and interact with borate in the manner described above.

The polysaccharides that may be utilized in the present invention include galactans, mannans, xylans, arabinans, rhamanans, and combinations thereof. The preferred polysaccharides have β-1,4 linked sugar backbones with a limited degree of branching. The preferred molecular weight range is greater than 10,000 Daltons, particularly 10,000 to 10,000,000 Daltons.

The preferred polysaccharides are galactans and mannans. Glucomannans are particularly preferred.

Glucomannans have a backbone that contains glucose and mannose subunits. The glucomannans are available from and obtained from various types of plants, such as Konjac. The structure of the compounds may be branched or linear, and both the glucose/mannose ratio and the sequence of glucose and mannose ratios may be varied. The molecular weights of the glucomannans utilized in the present invention may widely vary, but the molecular weights will generally be in the range of from about 50,000 to about 1,000,000 Daltons.

A particularly preferred glucomannan is commercially available from root of Konjac plant. It has glucose and mannose subunits with β-1,4 linkages at a molar ratio of 1.0:1.6, and is slightly branched (i.e., every 50 to 60 units) via a C₃ bond on hexoses of the main chain. Acetyl groups, which are located along the glucomannan backbone every 9 to 19 sugar units, contribute to the aqueous solubility of the compound. It has molecular weights of 200,000 to 2,000,000 Daltons. It is a food thickener and is commercially available from FMC corp.

Glucomannan isolated from Aloe Vera, commonly known as “Acemannan”, may also be utilized in the present invention. It is commercially available and is believed to be the main ingredient responsible for the wound healing effect of Aloe.

Other mannans with a low degree of branching may also be utilized in the present invention. For example, mannans that are produced by partial hydrolysis of galactomannans (e.g., by enzymatic hydrolysis of guar gum) are commercially available from Carbomer, Inc, San Diego, Calif.

The borate compounds which may be used in the compositions of the present invention are boric acid and pharmaceutically acceptable salts thereof, such as sodium borate (borax) and potassium borate. As used herein, the term “borate” refers to boric acid and all pharmaceutically suitable salts of boric acid. Borates are common excipients in ophthalmic formulations due to good buffering capacity at physiological pH and well known safety and compatibility with a wide range of drugs and preservatives. Borates also have inherent bacteriostatic and fungistatic properties, and therefore aid in the preservation of the compositions.

The compositions of the present invention will contain one or more polysaccharides and one or more borates in an amount sufficient to form a gel or partial gel when the composition is applied to the eye. The amount of polysaccharide and borates required for a particular composition will be determined based on various factors, such as the molecular weight and/or grade of the particular polysaccharide selected and the type of gelling properties desired.

The borate or polysaccharide concentration may be manipulated in order to arrive at the appropriate viscosity of the composition upon gel activation (i.e., after administration to the eye). If a strongly gelling composition is desired, then the borate or polymer concentration may be increased. If a weaker gelling composition is desired, such as a partially gelling composition, then the borate or polysaccharide concentration may be reduced. Other factors may influence the gelling features of the compositions of the present invention, such as the nature and concentration of additional ingredients in the compositions, e.g., salts, preservatives, chelating agents and so on.

The preferred non-gelled compositions of the present invention, i.e., compositions not yet gel-activated by the eye, will generally have a viscosity of from about 5 to 1000 cps. The preferred gelled compositions of the present invention, i.e., compositions gel-activated by the eye, will generally have a viscosity of from about 50 to 50,000 cps.

The compositions of the present invention will typically contain one or more polysaccharides in an amount of from about 0.1 to 5% weight/volume (“w/v”), and borate in an amount of from about 0.05 to 5% (w/v). Preferably, the compositions will contain 0.2 to 2.0% (w/v) of one or more polysaccharides and 0.1 to 2.0% (w/v) of a borate compound. Most preferably, the compositions will contain 0.3 to 0.8% (w/v) of one or more polysaccharides and 0.25 to 1.0% (w/v) of a borate compound.

The polysaccharide/borate gelling characteristics described herein can be customized by using a second polymeric material, such as povidone or cellulose derivatives (e.g., HEC, HPMC and others). Alternatively, non-polymeric polyols such as mannitol or sorbitol can be incorporated to limit the gel forming ability of a composition. The compositions of the present invention can additionally contain one or more antimicrobial agents to preserve the composition from microbial contamination, as well as essential ions found in human tears. Conditioning or comfort drop compositions for contact lenses according to this invention may additionally contain one or more surfactants to remove deposits from contact lenses.

Combinations of the gelling system of the present invention and prior gelling systems is also contemplated by the present invention. Such prior gelling systems may include ionomers, such as xanthan, gellan, carageenan and carbomers, and thermogels, such as ethylhydroxyethyl cellulose.

Other ingredients may be added to the compositions of the present invention. Such ingredients generally include tonicity adjusting agents, chelating agents, active pharmaceutical agent(s), solubilizers, preservatives, pH adjusting agents and carriers. Other polymer or monomeric agents such as polyethylene glycol and glycerol may also be added for special processing. Tonicity agents useful in the compositions of the present invention may include salts such as sodium chloride, potassium chloride and calcium chloride; non-ionic tonicity agents may include propylene glycol and glycerol; chelating agents may include EDTA and its salts; solubilizing agents may include Cremophor EL® and tween 80; other carriers may include Amberlite® IRP-69; pH adjusting agents may include hydrochloric acid, Tris, triethanolamine and sodium hydroxide; and suitable preservatives may include polyquaternium-1 and polyhexamethylene biguanide. The above listing of examples is given for illustrative purposes and is not intended to be exhaustive. Examples of other agents useful for the foregoing purposes are well known in ophthalmic formulation and are contemplated by the present invention.

The compositions of the present invention may be used to lubricate the eye or provide artificial tear solutions to treat, for example, dry eye. In general, artificial tear solutions will contain tonicity agents, polymers and preservatives, as described above.

The compositions of the present invention are primarily adapted for use as artificial tears or ocular lubricants. However, the compositions may also be utilized to administer various pharmaceutically active compounds to the eye. Such pharmaceuticals may include, but are not limited to, anti-hypertensive, anti-glaucoma, neuro-protective, anti-allergy, muco-secretagogue, angiostatic, anti-microbial, pain relieving and anti-inflammatory agents.

Examples of pharmaceutically active agents which may be included in the compositions of the present invention, and administered via the methods of the present invention include, but are not limited to: glaucoma agents, such as betaxolol, timolol, pilocarpine, carbonic anhydrase inhibitors and prostaglandins; dopaminergic antagonists; post-surgical antihypertensive agents, such as para-amino clonidine (apraclonidine); anti-infectives, such as ciprofloxacin and tobramycin; non-steroidal and steroidal anti-inflammatories, such as naproxen, diclofenac, suprofen, ketorolac, tetrahydrocortisol and dexamethasone; proteins; growth factors, such as epidermal growth factor; and anti-allergics.

The following Examples are provided to further illustrate the present invention:

### Example 1

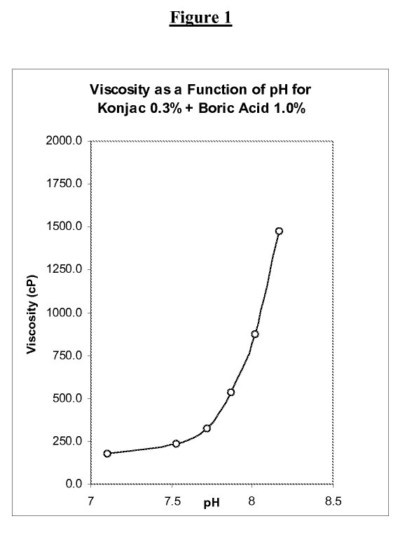

The viscosity versus pH of a composition containing a gelling system in accordance with the present invention was evaluated. The gelling system consisted of 0.3% Konjac glucomannan and 1.0% boric acid. The viscosity was measured as a function of pH. As shown in FIG. 1, the composition exhibited a strong ability to form gel as pH was increased, as demonstrated by the rapid increase in viscosity.

### Example 2

The following formulation is an example of an artificial tear composition of the present invention:

| | Concentration |
|---|---|
| Ingredient | (w/v %) |
| Konjac glucomannan | 0.25 |
| Boric Acid | 1.0 |
| Sodium Chloride | 0.1 |
| Potassium Chloride | 0.12 |
| Calcium Chloride | 0.0053 |
| Magnesium Chloride | 0.0064 |
| Zinc Chloride | 0.00015 |
| Polyquaternium-1 | 0.0005 |
| Sodium Hydroxide | pH to 6.5-7.0 |
| Purified Water | Qs to 100 |

The above composition is prepared in two parts. Konjac glucomannan is dispersed in 40% of the volume water and allowed to hydrate. The polymer solution is polish filtered and autoclaved at 122° C. for 30 minutes. The resulting solution (“Part I”) is then autoclaved at 121° C. for 35 minutes, and mixed while cooling. A second part (“Part II”) is prepared by dispersing the remaining ingredients in 40% of the batch volume of purified water and allowing the ingredients to dissolve and then adjusting the pH to near the target pH. The Part II solution is sterile filtered through a 0.2 micron sterilizing filter, and then aseptically added to Part I solution.

The above-described composition is a liquid in the bottle, which allows for ease of dispensing. Upon application of a small amount (e.g., 1 to 2 drops) of the composition to the eye, a soft fluid gel is formed upon slight increase in pH. The gel offers increased retention, relative to conventional ophthalmic solutions, and provides excellent lubrication to the eye.

We claim:

1. An ophthalmic composition that forms a gel or partial gel upon topical application of the composition to the eye of a human or other mammal, said composition containing an amount of polysaccharide/borate gelling system sufficient to facilitate the formation of said gel or partial gel, wherein the polysaccharide is not galactomannan and:
(i) has a structure that is predominately linear with less than one branched group per five sugar moieties;
(ii) contains cis-diol groups;
(iii) has α or β linkages at the 1,4-position within the sugar moieties; and
(iv) has a molecular weight of greater than 10,000 Daltons.

2. A composition according to claim 1, wherein said polysaccharide is glucomannan.

3. A composition according to claim 1, wherein said polysaccharide does not comprise galactan or mannan.

4. A composition according to claim 1, further comprising a therapeutically effective amount of a pharmaceutically active compound.

5. A method of delivering a pharmaceutically active compound to the eye, which comprises topically applying the composition of claim 4 to the affected eye.

6. A method of treating dry eye conditions, which comprises applying the composition of claim 1 to the eye.

7. An ophthalmic composition that forms a gel or partial gel upon topical application of the composition to the eye of a human or other mammal, said composition containing an amount of polysaccharide/borate gelling system sufficient to facilitate the formation of said gel or partial gel, wherein the polysaccharide is selected from the group consisting of:
galactans, xylans, arabinans, rhamanans, and combinations thereof.

8. A composition according to claim 7 wherein the polysaccharide is an arabinan.

9. A composition according to claim 8 wherein the arabinan is arabinogalactan.

10. A composition according to claim 8 wherein said arabinan does not comprise galactose or mannose.
